(19) United States
(12) Patent Application Publication
(10) Pub. No.: US 2018/0184950 A1
(43) Pub. Date: Jul. 5, 2018

(54) IMAGING DEVICE AND METHOD FOR DETECTION OF DISEASE

(71) Applicants: UNIVERSITY OF MIAMI, Miami, FL (US); VIGILANT BIOSCIENCES, INC., Ft. Lauderdale, FL (US)

(72) Inventors: Robert C. Bohannon, Elkhart, IN (US); Elizabeth Franzmann, Miami, FL (US); Matthew H.J. Kim, Parkland, FL (US)

(21) Appl. No.: 15/564,879

(22) PCT Filed: Apr. 6, 2016
(86) PCT No.: PCT/US2016/026223

§ 371 Date: Oct. 6, 2017

Related U.S. Application Data
(60) Provisional application No. 62/143,632, filed on Apr. 6, 2015.

(51) Int. Cl. A61B 5/1455; A61B 5/145; A61M 11/00; A61K 49/00; G01N 33/574; C07K 16/28

(52) U.S. Cl. CPC A61B 5/1455; A61B 5/14546; A61M 11/00; A61B 5/0071; A61K 49/0058; G01N 33/57407; C07K 16/2884; A61B 5/14507

(57) ABSTRACT

A system for testing a biological subject includes an imaging device and an imaging agent detectable by the imaging device, for detecting amount and location of a biomarker in the biological subject. The imaging agent includes a fluorescent microsphere and a targeting member attached to the fluorescent microsphere. A method of testing a biological subject using the system includes providing the imaging agent, applying the imaging agent to a testing location of a patient and imaging the imaging agent in the testing location by the imaging device, so as to detect the amount and locations of the imaging agent. A method of screening and diagnose a cancer that is accessible via cavity of a patient, includes performing a CD44 and total protein test, and if the result is positive, performing an imaging test.

### CROSS-REFERENCE TO RELATED PATENT APPLICATIONS

This application claims priority and the benefit of U.S. Provisional Application Ser. No. 62/143,632, filed on Apr. 6, 2015, entitled “Image Device and Method for Detection of Diseases,” the disclosure of which is incorporated herein in its entirety by reference.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE INVENTION

The invention relates generally to a system for detection diseases, and more specifically related to an imaging agent for detection of disease status.

### BACKGROUND OF THE INVENTION

The background description provided herein is for the purpose of generally presenting the context of the invention. Work of the presently named inventors, to the extent it is described in this background section, as well as aspects of the description that may not otherwise qualify as prior art at the time of filing, are neither expressly nor impliedly admitted as prior art against the invention.

In one aspect, oral cancer has high mortality. Oral squamous cell carcinomas (including those of the mouth and oropharynx) comprise more than 90% of all cancers affecting these tissues [1]. Of all the major cancers, oral cancer has one of the worst five-year survival rates at 62.7% [2]. Historically, the death rate associated with this cancer is particularly high because it is discovered late (stage III or IV) [1], often when it has spread to the lymph nodes or deeper structures. Late stage cancers have high mortality, are expensive to treat and have high morbidity [3-14]. Oral cancer is particularly dangerous, because in its early stages it is unnoticeable and can thrive without producing pain or recognizable symptoms or masquerade as innocuous infection or irritation, resulting in treatment delays [1]. Moreover, patients who survive a first encounter with the disease have up to a 20 times higher risk of developing a second oral or esophageal cancer [15].

In another aspect, oral cancer is expensive to treat. Oral cancer is more than 3 times as common as cervical cancer [2] with over 40,000 Americans diagnosed annually resulting in well over 8,000 expected deaths in 2014 [2]. Over 600,000 new cases of head and neck cancer are diagnosed worldwide each year, the majority of which are oral cancer [16]. In practice, 1.08% of men and women born today will be diagnosed with this cancer sometime during their lifetime [2]. The vast majority of oral cancers occur in people older than 45 years, with men nearly three times as likely as women to develop the disease [2, 17]. The main risk factors are tobacco, alcohol, and human papillomavirus infection [18-28). The direct medical costs for head and neck cancer in the US in 2010 totaled an estimated $3.64 billion [6]. Commercially-insured individuals revealed that the average medical costs of oral and salivary gland cancers in the first year after diagnosis was $79,151, which is significantly higher than the cost to treat other cancers ($31,559-$65,123) [7-9]. Worldwide, oral cancer is the costliest cancer in low-income countries [29).

The current standard of care for oral cancer screening and diagnosis is a physical and visual examination followed by biopsy. Typically, a patient notices a lesion after it becomes painful or interferes with speech or swallowing. Occasionally, their dentist may be the first to notice it. The patient may be treated with antibiotics for a period of time, particularly if they initially present to a primary care physician. Eventually, they are sent for biopsy and pathologic diagnosis. Unfortunately, due to these delays most cases are diagnosed in late stage (III or IV) with a five-year survival rate around 30-40% even after aggressive treatment regimens including combinations of radiation, surgery and chemotherapy [1]. However, if diagnosed early (stage I/II), the five-year survival-rate can be as high as 80-90% and represent a significant healthcare cost savings [1,7]. A survey conducted by the ADA revealed that only 16% of patients reported having an oral cancer examination during a routine dental appointment or were unaware that the screening had been performed [1,28]. Failure to perform a routine oral cancer screening or early diagnostic test has unintentionally placed the responsibility on the dental patient to alert the medical professional about the presence of an oral lesion, which is often too late.

Thus, there is a pressing need for an improved early detection test targeting cancers accessible via cavity such as oral, lung, esophagus, cervical, anal, and colorectal cancer, which will save lives, improve clinical outcome and reduce treatment costs.

### SUMMARY OF THE INVENTION

In one aspect, the invention related to a system for testing a biological subject. In one embodiment, the system includes an imaging device and an imaging agent detectable by the imaging device. The imaging agent includes a fluorescent microsphere and a targeting member attached to the fluorescent microsphere.

In certain embodiments, the targeting member is an antibody or an aptamer. In one embodiment, the antibody specifically binds CD44.

In certain embodiments, the imaging device is configured to detect light in a range of about 400-700 nm corresponding to the property of the imaging agent. In certain embodiments, the fluorescent microsphere includes a green dye having an excitation maxima at 480 nm and an emission maxima at 520 nm. In certain embodiments, the fluorescent microsphere includes a blue dye having an excitation maxima at 360 nm and an emission maxima at 450 nm.

In certain embodiments, a particle size of the fluorescent microsphere is in a range of 0.2 μm-15 μm. In certain embodiments, the particle size of the fluorescent microsphere is in a range of 0.2 μm-0.22 μm.

In certain embodiments, the fluorescent microsphere has carboxyl groups and the targeting member is covalently linked to the fluorescent microsphere through the carboxyl groups. In certain embodiments, the fluorescent microsphere is made of latex and the targeting member is adsorbed to a surface of the fluorescent microsphere through hydrophobic attractions.

In certain embodiments, during testing of the biological subject, the imaging agent is used to rinse the mouth of a patient and is then spit by the patient, and the imaging agent bound to a target location in the mouth is detected by the imaging device. In certain embodiments, during testing of the biological subject, an ultraviolet light or any light that is capable of exciting the fluorochrome to emit in the visible range is applied to the mouth of the patient.

In certain embodiments, during testing of the biological subject, the imaging agent is sprayed to a test location of a patient, and then imaged using the imaging device.

In another aspect, the present invention relates to a method of testing a biological subject. In one embodiment, the method includes:

providing an imaging agent, the imaging agent comprising:
a fluorescent microsphere; and
a targeting member attached to the fluorescent microsphere;

rinsing a testing location of a patient using the imaging agent;

spitting the imaging agent; and

imaging, by an imaging device, the imaging agent in the testing location, so as to detect an amount and a position of the imaging agent in the testing location.

In certain embodiments, the targeting member is an antibody or an aptamer. In one embodiment, the antibody specifically binds CD44.

In certain embodiments, the imaging device is configured to detect light in a range of about 400-700 nm corresponding to the property of the imaging agent. In certain embodiments, the fluorescent microsphere includes a green dye having an excitation maxima at 480 nm and an emission maxima at 520 nm. In certain embodiments, the fluorescent microsphere includes a blue dye having an excitation maxima at 360 nm and an emission maxima at 450 nm.

In certain embodiments, a particle size of the fluorescent microsphere is in a range of 0.2 μm-15 μm. In certain embodiments, the particle size of the fluorescent microsphere is in a range of 0.2 μm-0.22 μm.

In certain embodiments, the fluorescent microsphere has carboxyl groups and the targeting member is covalently linked to the fluorescent microsphere through the carboxyl groups. In certain embodiments, the fluorescent microsphere is made of latex and the targeting member is adsorbed to a surface of the fluorescent microsphere through hydrophobic attractions.

In certain embodiments, the step of applying the imaging agent to the test location of the patient includes rinsing the test location with the imaging agent or spray the imaging agent on the test location.

In a further aspect, the present invention relates to a method of screening and diagnose a cancer that is accessible via cavity of a patient. In one embodiment, the method includes the steps of: performing a test measuring a biomarker selected from a group consisting of at least CD44, total protein, and both CD44 and the total protein; and if a result from the performing the test is positive, performing an imaging test.

In certain embodiments, the step of performing the test includes: measuring a concentration of CD44 in a sample from the patient; and measuring a concentration of total protein in the sample.

In certain embodiments, the result of the step of performing the test is positive when the concentration of CD44 in the sample is greater than a first pre-determined threshold and the concentration of the total protein in the sample is greater than a second pre-determined threshold.

In certain embodiments, the step of performing an imaging test includes:

providing an imaging agent, the imaging agent comprising:
a fluorescent microsphere; and
a targeting member attached to the fluorescent microsphere;

applying the imaging agent to a test location; and

imaging, by an imaging device, the imaging agent in the testing location, so as to detect an amount and a position of the imaging agent in the testing location.

In certain embodiment, the step of applying the imaging agent includes rinsing the test location with the imaging agent or spray the imaging agent on the test location.

In certain embodiments, the targeting agent is an antibody or a aptamer. In certain embodiments, the imaging device is configured to detect light in a range of about 400-700 nm corresponding to the property of the imaging agent. In certain embodiments, the antibody specifically binds CD44, the fluorescent microsphere includes a green dye having an excitation maxima at 480 nm and an emission maxima at 520 nm or a blue dye having an excitation maxima at 360 nm and an emission maxima at 450 nm, and a particle size of the fluorescent microsphere is in a range of about 0.2 μm-0.22 μm.

In certain embodiments, the fluorescent microsphere has carboxyl groups and the antibody is covalently linked to the fluorescent microsphere through the carboxyl groups.

Further areas of applicability of the invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The following figures form part of the present specification and are included to further demonstrate certain aspects of the invention. The invention may be better understood by reference to one or more of these figures in combination with the detailed description of specific embodiments presented herein. The drawings described below are for illustration purposes only. The drawings are not intended to limit the scope of the present teachings in any way.

FIG. 1 shows schematically a system for testing a biological subject according to one embodiment of the present invention.

FIG. 2 shows schematically an imaging agent according to one embodiment of the invention.

FIGS. 3A-3C show distribution of CD44 in different type of cells according to one embodiment of the present invention, where FIG. 3A shows result of normal mucosa cells, FIG. 3B shows result of dysplasia cells, and FIG. 3C shows result of invasive cancer cells.

FIG. 4A shows an image of a testing square (chip) according to one embodiment of the present invention, where a serial dilution of CD44 antibody, negative control of BSA, and positive control of goat anti mouse antibody (GaM), were attached to the Dragon Green beads and tested.

FIG. 4B schematically shows placement of different samples on a testing square (chip) for FIG. 4C and FIG. 4D, according to one embodiment of the present invention.

FIG. 4C shows comparison between Dragon Green and Glacial Blue microspheres according to one embodiment of the present invention.

FIG. 4D shows imaging results of using Glacial Blue microspheres according to one embodiment of the present invention.

FIG. 4E shows imaging results of using Quantum dots (QD) according to one embodiment of the present invention.

FIG. 5 shows schematically a method for testing a biological subject according to one embodiment of the present invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used herein, the term “antibody” refers to a polypeptide ligand substantially encoded by an immunoglobulin gene or immunoglobulin genes, or fragments thereof, which specifically binds and recognizes an epitope (e.g., an antigen).

As used herein, the term “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase the enzyme-linked immunosorbent assay (ELISA) are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

As used herein, the term “subject”, “patient” or “individual” generally refer to a human, although the methods of the invention are not necessarily limited to humans, and should be useful in other mammals.

As used herein, the term “CD44” is intended to include soluble CD44 and isoforms thereof.

The description will be made as to the embodiments of the invention in conjunction with the accompanying drawings. In accordance with the purposes of this invention, as embodied and broadly described herein, this invention, in one aspect, relates to a system and a method for testing a biological subject.

In one aspect, the present invention relates to a method for evaluating risk of cancer of a patient. In certain embodiments, a unique, easy to use, oral cancer specific, rapid lateral flow point-of-care (POC) test is provided. The result indicates if a patient has a high risk or a low risk of having cancer. The determination of the CD44 and the total protein may be implemented by a test strip and a colorimetric pad, respectively. The sample may be saliva (or an oral rinse capturing saliva, among other substances) from the patient, and the amount of the CD44 and the total protein is used to accurately assess patient's risk for and aid in the diagnosis of a cancer. In certain embodiments, the cancer may be a cancer that is accessible via cavity such as oral, lung, esophagus, cervical, anal, and colorectal cancer. In certain embodiments, the above described test system may identify those individuals at most risk of harboring early or developing oral cancer from the 85 million tobacco and alcohol users.

However, often these early lesions can be invisible to the naked eye, so the test strip alone may not indicate where the abnormal cells are in cases of very early disease. Accordingly, in another aspect, the present invention relates to a system and a method for testing a biological subject, where risk of cancer or lesions of a patient can be imaged and located accurately.

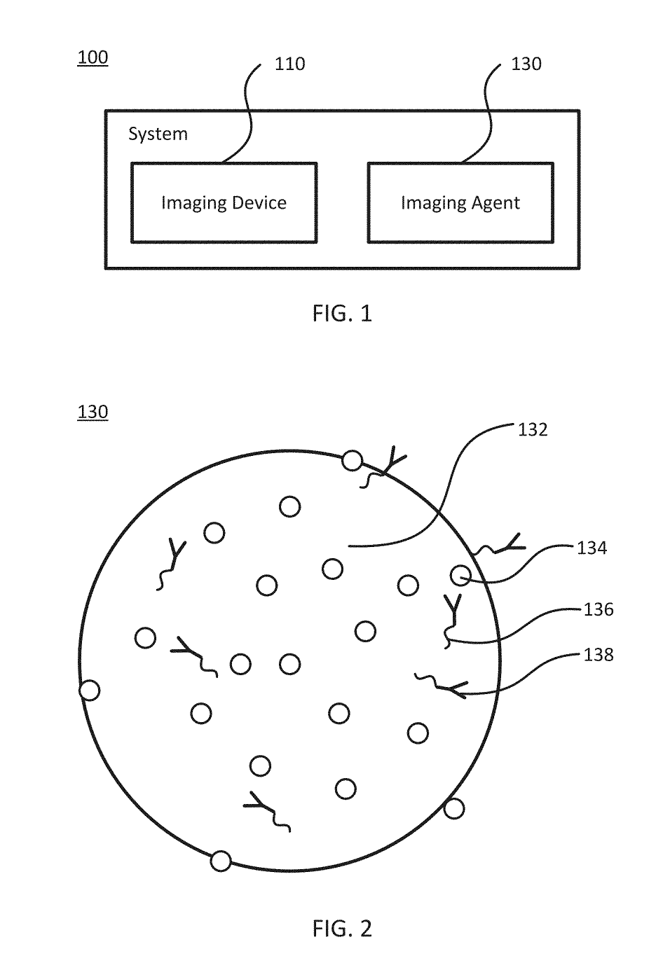

In certain embodiments, as shown in FIG. 1, a system 100 for testing a biological subject includes an imaging device 110 and an imaging agent 130. The imaging agent 130 may be provided as a simple rinse and spit solution, or may be provided as a spray for spraying the imaging agent towards the testing area of a patient. The imaging agent 130, when applied to the patient, may specifically bind certain target areas in the mouth of the patient, such as cancer cells, cells having high risk of becoming cancer cells, or cells having lesion that related to cancer. Then, the imaging agent 130 distributed in the targeted areas is detected by the imaging device 110, so as to determine the specific locations of the target areas and evaluate the lesion of the target areas. In certain embodiments, the imaging device 110 is configured to detect light in a range of about 400-700 nm corresponding to the property of the imaging agent.

FIG. 2 schematically shows a structure of the imaging agent 130 according to one embodiment of the present application. As shown in FIG. 2, the imaging agent 130 may include a microsphere 132, an imaging member 134, a linking member 136, and a functional member 138.

In certain embodiments, the diameter of the microsphere 100 is greater than 50-100 nm, such that the microsphere 100 is not absorbed into the bloodstream of a patient. In certain embodiments, the microsphere 100 has a diameter of about 0.10-30 μm. In one embodiment, the diameter of the microsphere 100 is about 0.20-15 μm. In one embodiment, the diameter of the microsphere 100 is about 0.20-2 μm. In one embodiment, the diameter of the microsphere 100 is about 0.20-0.22 μm.

In certain embodiments, the microsphere 100 is in a perfect sphere shape. In other embodiments, the microsphere 100 may not be in a perfect sphere shape. The microsphere 100 may have an oval shape, a polyhedron shape, or an irregular shape.

In certain embodiments, the microsphere 100 is made of polystyrene. The microsphere may also be made of other materials, as long as it is inert and does not react with the molecules in the patient, and does not interfere with the fluorescent signal. In certain embodiments, the microspheres 100 are quantum dots (Q dots, or QD).

The imaging member 134 may be disposed at the surface of the microsphere 132, or disposed both at the surface and the inside of the microsphere 132. In certain embodiments, the imaging member 134 may be fluorescent molecules. In certain embodiments, the imaging member 134 is configured to be detected by an ultraviolet (UV) light. In certain embodiments, the imaging member includes enzymes, biotin, streptavidin and fluorochromes covering the spectrum from UV to far infrared.

In certain embodiments, conjugates of the imaging member 134 and the functional member 138 may be used in a large number of scientific applications including Western Blotting, immunofluorescence, immunohistochemistry, flow cytometry, ELISA and Førster resonance energy transfer (FRET). In certain embodiments, the imaging member 134 may be fluorescein that has an absorption maxima at about 494 nm and an emission maxima at about 512 nm. In one embodiment, the imaging member 134 is fluorescein isothiocyanate (FITC). In certain embodiments, the imaging member 134 may be excited by UV light, such as is used by dentists' tools.

The microsphere 132 and the imaging member 134 may together form an imaging microsphere. The imaging member 134 may be disposed at the surface of the microsphere 132, or disposed both at the surface and the inside of the microsphere 132. In one embodiment, the imaging member 134 is substantially evenly distributed all through the microsphere 132. In certain embodiments, the imaging member 134 is impregnated into the microsphere 132.

In certain embodiment, the imaging member 134 is a fluorescent compound, and the imaging microsphere, including the microsphere 132 and the imaging member 134, is a fluorescent microsphere. In one embodiment, the imaging microsphere may be latex microspheres (beads) that do not include a specific linking member 136. The terminal group of the latex imaging microsphere may function as the linking member, and the functional member 138 may be absorbed to the surface of the imaging microsphere through, for example, hydrophobic interaction. In one embodiment, the fluorescent microspheres are non-functionalized microspheres that are suitable for coating via adsorption. The fluorescent microsphere may be a ˜1% solids (w/v) aqueous suspensions. In one example, the fluorescent microsphere is the fluorescent polymer manufactured by Bangs Laboratories, Inc. (Fishers, Ind., U.S.), the size of the microspheres is bout 0.2-0.22 μm, and the dye of the microspheres is Glacial Blue (excitation maxima at 360 nm, and emission maxima at 450 nm) or Dragon Green (excitation maxima at 480 nm, and emission maxima at 520 nm).

The linking member 136 is substantially disposed on the surface of the microsphere 132, and used to link the functional member 138 to the microsphere 132. The linking member 136 may be a carboxyl group, and the functional member 138 may be covalently linked to the linking member 136. In one embodiment, the microspheres are fluorescent microspheres have carboxyl functional group 136. Biomolecules may be covalently immobilized to carboxyl-functionalized microspheres. Fluorescent carboxyl microspheres may be available as ˜1% solids (w/v) aqueous suspensions. In one example, the fluorescent microsphere is the fluorescent carboxyl polymer manufactured by Bangs Laboratories, Inc. (Fishers, Ind., U.S.), the size of the microspheres is bout 0.2-0.22 μm, and the dye of the microspheres is Glacial Blue (360, 450) or Dragon Green (480, 520). In other words, the microsphere of Bangs Laboratories, Inc. (Fishers, Ind., U.S.) includes the microsphere 132, the imaging member 134, and the linking member 136.

The number or concentration of the linking member 136 on the surface of the microsphere 132 may be configured such that it provides sufficient biding activity for the functional member 138, while not affecting the imaging effect of the imaging member 134. For example, the linking member 136 may cover about 10-90% of the outer surface of the microsphere 132. In certain embodiments, the linking member 136 may cover about 30-70% of the outer surface of the microsphere 132. In certain embodiments, the linking member 136 may cover about 45-55% of the outer surface of the microsphere 132. In certain embodiments, the linking member 136 may cover about 50% of the outer surface of the microsphere 132.

The functional member 138 is attached to the surface of the microsphere 132 via the linking member 136 or attached directly to the microsphere having the imaging member 134. The functional member 138 is able to interact with certain target areas of the patient. In certain embodiments, the functional member 138 is a targeting member including an antibody or an aptamer, which specifically bind or interact with a target area of the patient. For example, the functional member 138 may be an anti-CD44 antibody, and the microsphere 132 and the imaging agent 134 form a fluorescent microsphere. The imaging agent 130, via the anti-CD44 antibody, is able to bind to cells expressing abnormally high levels of CD44.

When the functional member 138 is bound to the target area, that is, the imaging agent 130 is attached to the target area, the bound fluorescent microsphere may then be visualized by shining a light of specific wavelength. In certain embodiments, the specific wavelength of the light correspond to the feature of the imaging agent 130 used. The working wavelength of the light may be in a range of about 400-700 nm. In one embodiment, the imaging agent 130 includes Glacial Blue and the wavelength of the light used is about 390-395 nm. In another embodiment, the imaging agent 130 includes Dragon Green and the wavelength of the light used is about 460 nm. The imaging agent 130 may be identified by the imaging device 110 via viewing of the imaging member 134, so that very early lesions can be identified.

In certain embodiments, the microsphere 132 and the linking member 136 may not be necessary, and the functional member 138 is attached directly to the imaging agent 134 to form the imaging agent 130.

A variety of biomarker-based technologies exist to detect cancers but each has major drawbacks. Several studies have tested saliva for RNA expression profiles, microRNA discovery and proteomic analysis [32-37]. However, none of these are linked to an imaging modality. Light-based, spectroscopic and chemiluminescent adjuncts to physical exam became clinically available in recent years and attempt to address the need for earlier intervention. However, widespread acceptance of these light-based diagnostic tools has been far from optimal (Table 1).

Extensive searches of National Institutes of Health (NIH) and Small Business Innovation Research (SBIR) databases revealed only a few technologies under development that relate to oral cancer detection. Some emerging technologies are related to imaging techniques for oral cancer detection [5, 30-37, 39, 40], but most of these are rather nonspecific for cancer and use differences in reflected light and impedance between normal and abnormal tissues. For example, HN1 is a peptide designed to home in to head and neck cancer to help surgeons locate a cancer. However it is not linked to a companion risk assessment marker and is in very early stages [41, 42]. Another technology uses the lectin WGA labeled with fluorochrome sprayed on to mucosal surface and light is shown. Suspicious areas can be painted with dye to better delineate for treatment, but again there is no companion risk screening device or diagnostic aid to identify those who would most benefit from this technology [43, 44]. Because of its accuracy, simplicity, and low cost, the OncAlert products measuring CD44 together with total protein according to certain embodiments from Vigilant Biosciences (Fort Lauderdale, Fla., U.S.), stands alone as a front line screening tool and diagnostic aid for the millions at risk for this terrible disease. Table 1 shows a comparison of the OncAlert CD44+Total Protein product lines according to certain embodiments and related technique of other light systems.

TABLE 1
Comparison of CD44+Total Protein verses other technologies*.

| Detection Technique (reference) | Sensibility | Specificity |
|---|---|---|
| CD44 + Total Protein | 0.88 | 0.74-0.95 |
| Standard Visual/Physical Oral Exam (5) | 0.64 | 0.74 |
| Toluidine (Dye) (31) | 0.40 | 0.90 |
| Chemiluminescence (ViziLite) (30) | 0.00 | 0.76 |
| Autofluorescence (30) | 0.50 | 0.39 |

*The studies selected involved the screening of individuals without high-risk lesions and used biopsy as gold standard.

In certain embodiments, the imaging system of the present invention, among other things, has the following beneficial advantages.
1. The imaging system may be paired with companion risk assessment tool or diagnostic aid to identify subjects or patients that having tumor cells overexpressing CD44, high levels of total protein, or both.
2. CD44 is required for tumor initiation and therefore is overexpressed in very early cancer but not in other benign conditions. The imaging agent or a probe according to certain embodiments of the present invention, is linked to tumor initiating marker CD44. Therefore, early detection of tumor is achieved easily by the CD44 specific imaging agent and the corresponding imaging device.
3. The present invention may be performed simply and noninvasively in doctor or dentist's office by rinse and spit.
4. The present invention may be used to pinpoint region of carcinogenesis for therapeutic targeting.

These and other aspects of the present invention are more specifically described below.

### Examples

Without intent to limit the scope of the invention, exemplary system and method according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1: CD44 and Total Protein as an Indicative of Cancer

In certain embodiments of the present invention, the combination of the markers CD44 and total protein in oral rinses effectively distinguished subjects with head and neck squamous cell carcinoma (HNSCC) from those without the disease. According to certain embodiments of the present invention, an ELISA plate was used that recognized all soluble CD44 (solCD44) normal and variant isoforms (total solCD44). The total solCD44 levels were statistically higher in HNSCC cases compared to controls. Conversely, there were no significant elevations noted in subjects with benign disease of the upper-aerodigestive tract indicating that the marker was specific for cancer. Total protein, measured with a simple Lowry-like assay was added as a marker since it costs only pennies per sample and improves risk stratification. In certain embodiments, other tests to measure total protein known to those of ordinary skill in the art may be used. The combined test (high total CD44 and high protein) was associated with much higher risk (Odds Ratio (OR) 25, p<0.0001) than the reference group while high levels of either total protein or CD44 were associated with OR of 2.8 (p=0.006) and 8.9 (p<0.0001), respectively [14].

### Example 2: Case-Control Hospital-Based Study to Develop Cutpoints for Oral Rinse Test

In order to further develop the test and determine clinically significant cut points, we evaluated 150 oral cancer patients from 150 controls which were frequency matched for age, gender, race, ethnicity, tobacco and alcohol use and socioeconomic status (SES). Using these, subjects and multivariate recursive partitioning, 2 cut-points each for protein and CD44 were identified that separated cases and controls into specific risk groups. These cutpoints were validated in another small case-control group. Then the specificity was determined using 150 controls with a history of tobacco and/or alcohol use. Sensitivity and specificity for distinguishing stage I-III cases from these controls was 88% and 95% when control patients were followed over time. This test has been converted to a lateral flow system that measures solCD44 and protein levels in oral rinses. Positive results mean the subject is at significantly higher risk for oral cancer than subjects with similar risk factors but normal marker levels.

### Example 3: Design of the Imaging Agent and Testing CD44 on Membranes

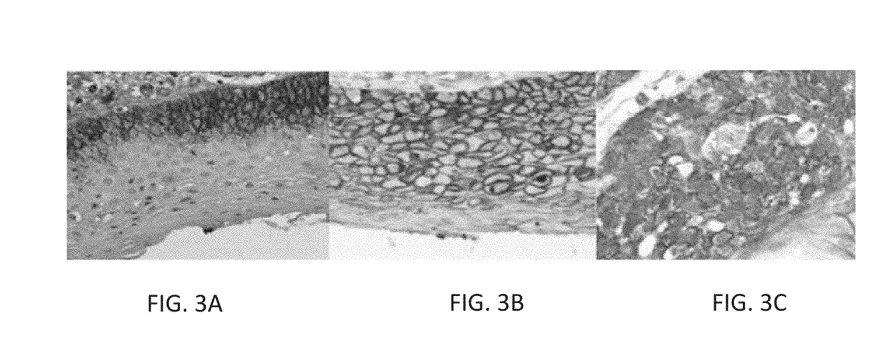

While this lateral flow test strip described in Example 2 accurately identifies individuals at most risk for cancer, it does not identify where the cancerous cells triggering abnormal marker levels are located. FIGS. 3A-3C shows respectively the CD44 expression in normal cell, in dysplasia cell, and in squamous cell carcinoma. CD44 is expressed ubiquitously in many tissues, however, as shown in FIGS. 3A-3C, CD44 is present in the basal and suprabasal epithelial layers in normal mucosa, but the expression extends to the superficial layers, which would have contact with the oral rinse, in dysplasia and invasive cancer. In other words, the CD44 expression is limited to the more basally located levels of the epithelium, whereas in dysplasia the expression involves all of the epithelial layers, and in cancer it can be expressed throughout the tumor. According to the above described results, an oral rinse consisting of an anti-CD44 antibody conjugated to fluorescently-labeled non-absorbable microspheres is designed to contact and bind to those areas of the epithelium, where CD44 is abnormally expressed. The fluorescent label is then permit visualization of the lesion location.

Fluorescently labeled microspheres are increasingly seen in clinical use and research with reported uses in inflammatory bowel diseases such as ulcerative colitis and dental imaging. Their advantage is that they are inert and not absorbed due to their size. Thus if ingested they are simply excreted as waste as occurs with Kayexalate®, which is a polystyrene neutralized with sodium ions that is used to chelate potassium in hyperkalemic patients [45-47].

To develop an imaging agent for CD44-expressing oral squamous cell dysplasia and carcinoma, multiple types of fluorescent beads and multiple different anti-CD44 antibodies were tested. The multiple types of fluorescent beads and multiple different anti-CD44 antibodies were applied to a membrane (nitrocellulose) with known quantities of CD44 spotted in specific locations and non-human protein negative control and anti-antibody control (to determine if coupling of the fluorescent beads and antibodies was successful). Then a 400-460 nm ultraviolet (UV) light is used to visualize the resulting spots. In certain embodiments, 8 different latex beads, 4 anti-CD44 antibodies, and over 2 dozen diluent (mouthwash representative) conditions were tested.

### 3.1 Latex Bead Label

In certain embodiments, it was shown that the different latex microspheres (beads) with various sizes and types could be spotted (unconjugated) with strong signals under UV light (1% solids in aqueous solution). In certain embodiments, the fluorescently labeled polystyrene beads were obtained from Bangs Laboratories, Inc. (Fishers, Ind., U.S.). Six of these fluorescently labeled polystyrene beads were “Dragon Green” (480/520 excitation/emission maxima), and 2 others were Glacial Blue (360/450 excitation/emission maxima). Some of the beads were carboxyl latex beads, and others were non-covalent latex beads. The beads were approximately 0.2 μm to 15 μm particle-size, which is well over the 50-100 nm (0.05-0.1 μm) size that can be absorbed into the bloodstream [47].

In certain embodiments, non-carboxyl FITC beads of various sizes (in a range of about 0.2-15 μm) were tested. The fluorescently labeled polystyrene beads were non-covalent latex beads where the active agent is adsorbed via hydrophobic (Vander Waals, London Type) attractions between the ligands and the polymeric surface of the microspheres. However, antibodies may be difficult to couple to and little to no signal could be seen.

In certain embodiments, the fluorescently labeled polystyrene beads were carboxyl beads where the antibody was covalently linked to the microsphere. The 0.22 μm carboxyl latex FITC beads (covalent coupling) were used to successfully couple to 4 different CD44 antibodies, and at least 2 dozen different diluents were screened to dilute the carboxyl beads into for optimal signals.

Further, direct-labeled FITC to antibodies, as well as direct-labeled QDs to antibodies were tested. The direct-labeled FITC antibodies yielded very low response, and while the QDs were extremely vibrant in color (as was the FITC solution) under UV light, they did not couple to the antibodies well. Multiple pH conditions were checked, as were different antibodies.

In certain embodiments, larger-sized carboxyl FITC beads of both types (5-10 μm in size) did not work well as the 0.2 μm beads.

In certain embodiments, the carboxyl beads of smaller size (˜0.2 μm) for both Dragon Green and Glacial Blue worked well. As a result, one carboxyl Dragon Green microsphere and one carboxyl Glacial Blue microsphere were chosen for further analysis, both of which were 0.2-0.22 μm in size (the larger carboxyl bead sizes & non-carboxyl beads failed conjugate to the antibody well). The Glacial Blue FITC microspheres showed better high-end results, while the Dragon Green microspheres seemed to be slightly better at the lower concentrations of positive serum.

### 3.2 Anti-CD44 Antibodies:

Different anti-CD44 antibodies that had previously worked well in ELISA and lateral flow applications were chosen. The 0.22 μm Dragon Green beads were used on the antibodies tested. All of the antibodies appeared to give similar signal, and clone of one of the antibodies from a preferred supplier was used for further tests.

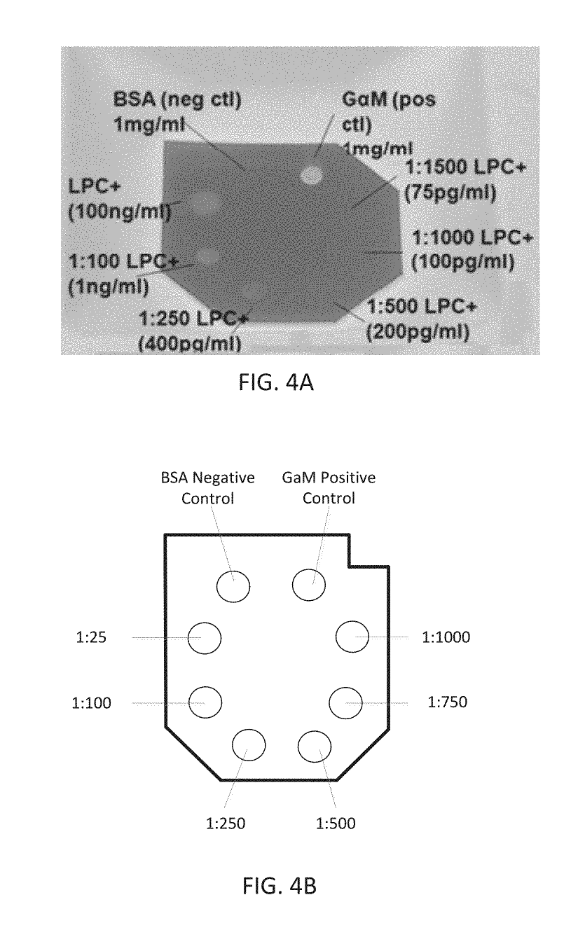

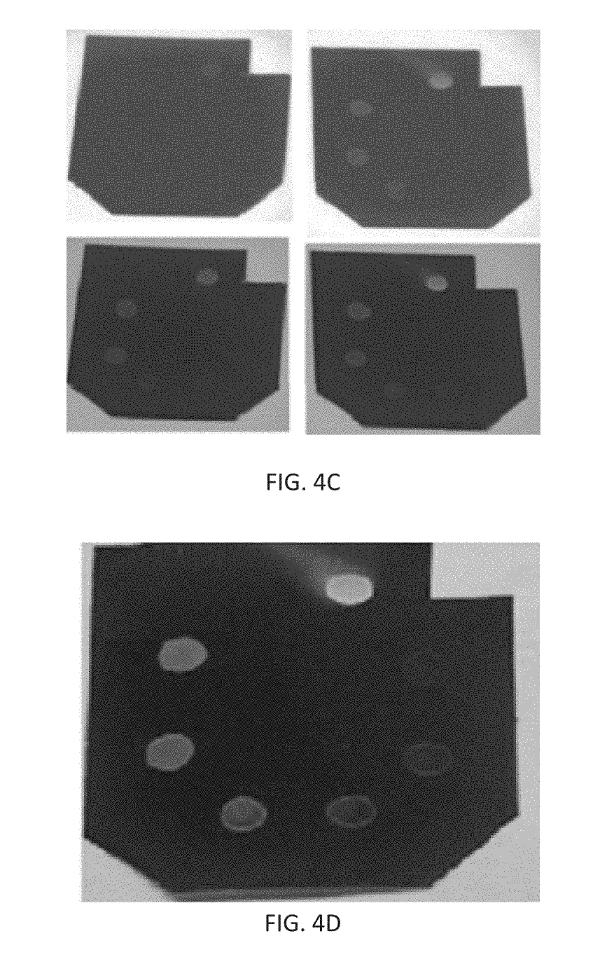

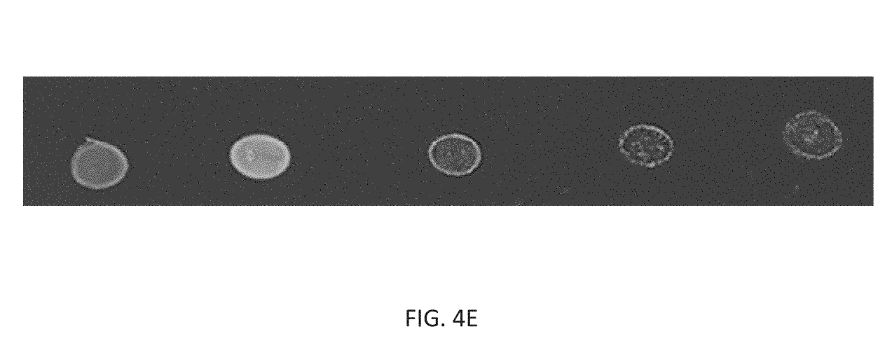

The aforementioned Glacial Blue 0.2 μm beads were also tested. While similar to Dragon Green beads in level of detection (LOD) and intensity, the Glacial Blue beads had slightly stronger signal with the UV lamp used (as well as easier to image without a filter on the camera, as seen in FIG. 4C, where top row=unfiltered camera, bottom row=yellow filter, left column=Dragon Green beads, right column=Glacial Blue beads).

Following further optimization, a discernable signal was seen easily at 1:750, and slightly at 1:1000 for the Glacial Blue beads, as shown in FIG. 4D.

In addition to the Dragon Green and the Glacial Blue carboxyl beads, 3 nontoxic QD formulations prior to conjugation were also tested. FIG. 4E shows Dragon Green, Glacial Blue, and three QDs sequentially without conjugation with antibody. Initial testing with the second QD formulation yielded similar results to the larger sized carboxyl beads (they did not conjugate to the antibody well), thus we decided to move forward with the anti-CD44 conjugated Dragon Green and Glacial Blue were chosen for further testing since they conjugated easily to the antibody giving detectable spots.

### 3.3 Diluent

The diluent was used to make dilutions of the stock solution of the CD44 conjugated beads. A 1:100 dilution is made with, for example, about 5 μL of stock solution diluted in 500 μL diluent. Over two dozen different diluents were tested, of which 3 were “finalists”. The difference between the three finalists were the surfactants used. The three final diluents are PBS with the surfactants Tween 20, Tergitaol, and F127, respectively, where the diluents containing Tergitol is better than the diluents containing F127 or Tween 20. The following images were collected using the best of these three formulations.

### 3.4 Testing CD44 on Membranes

Six different iterations of nitrocellulose “test squares” were developed, eventually using BSA at 1 mg/ml as a negative control, Goat anti-mouse at 1 mg/ml as a positive control, and a known CD44 positive serum at ˜100 ng/ml stock using dilutions down to 1:1000 (or roughly 100 pg/ml) to determine how well the various antibodies and diluents worked.

After antibody, diluents, and initial labels were chosen, at a first step, a test square (chip) is provided, which includes CD44 attached thereon. In certain embodiments, the square is a nitrocellulose membrane with multiple CD44 spots.

Then, at the second step, various concentrations of low CD44 positive control (LPC) were spotted on a test square (chip). The antibody-labeled Dragon Green carboxyl microsphere was applied at beads:diluent ratio of 1:25 (for example, 20 μL microspheres +500 μL diluent). As shown in FIG. 4B, the diluent includes: 1 mg/ml bovine serum albumin (BSA) as negative control, 1 mg/ml goat anti-mouse (GαM) antibody as positive control, and CD44 positive serum (CD44 antibody) at 100 ng/ml (stock), 1 ng/ml, 400 pg/ml, 200 pg/ml, 100 pg/ml, 75 pg/ml of LPC (corresponding to 1, 1:100, 1:250, 1:500, 1:1000, and 1:1500 dilution, respectively).

In the third step, after the beads/diluent mixture were applied to the test square, the test square was incubated to allow reaction between the antibody and the CD44 protein. In one example, the optimal incubation time was 10 minutes. Longer incubation yielded slightly more signal, which capped at about 30 minutes.

In the fourth step, after 10 minutes incubation, at room temperature, discard the mixture (or save, which showed to be as robust on a 2nd test during the investigation), then wash/rinse/discard 3 times with 1× phosphate buffered saline with Tween 20 (PBST).

In the fifth step, the test square was dried for 5 minutes at 37-45° C., and then was placed under a UV lamp of appropriate wavelength and shined under the UV lamp.

As a result shown in FIG. 4A, when UV light was applied, the limit of detection (LOD) was seen at LPC dilution at 1:500 or about 200 pg/ml. In certain embodiments, the yellow filter was used on the camera to improve the signal. In other embodiments, other type of filter may be used or the filter is not necessary, depending on the imaging agent and the wavelength of the UV light, and/or other conditions used.

Further, FIG. 4B is a legend showing the location and concentrations of the LPC for FIGS. 4C and 4D. As shown in FIG. 4B, in each of the plates of FIGS. 4C and 4D, LPC is spotted with a dilution of 1:25, 1:100, 1:250, 1:500, 1:750 and 1:1000, BSA was used as negative control, and GaM was used as positive control.

FIG. 4C shows comparison between Dragon Green and Glacial Blue microspheres. The Dragon Green is applied in the two left plates, and the Glacial Blue is applied in the two right plates. The top row plate image are collected under UV light using a camera without filter, and the bottom row plate images are collected under UV light using a camera with yellow filter. In each of the plates, LPC is spotted with a dilution in a range from 1:25 down to 1:1000. A faint band can be seen at LPC 1:750 which is equivalent to LOD of 150 pg/ml. To see the Dragon Green results better, a yellow filter was placed (bottom row of FIG. 4C), while Glacial Blue worked well without the yellow filter (the top row of FIG. 4C). As a result shown in FIG. 4C, no false positives were seen with BSA, and GaM positive control was very bright. The final LOD=1:750 of low positive control (˜100 ng/ml stock), or roughly 150 pg/ml. Longer incubation (30 minutes) yielded very faint signal at a ratio of 1:1000, but more confident at a ratio of 1:750. In one embodiment, it was shown that Glacial Blue is easier to photograph than the Dragon Green.

FIG. 4D shows results using Glacial Blue after further optimization with a spot now detected at 100 pg/ml (1:1000 LPC).

In addition, FIG. 4E shows (from left to right) 1 uL spots onto Nitrocellulose of Dragon Green and Glacial Blue carboxyl beads and three QDs formulations, unconjugated to antibody, starting at 10 mg/ml, then diluted 1:25 into the final diluent selected. Since these were unconjugated to antibody (to show maximum potential signal) there was no CD44 spotted for this initial screen. Both the latex beads and the QDs were able to shown colors of the fluorescent attached to the latex beads and the QDs. However, the signal intensity for the QDs appeared lower than the signal intensity of the latex beads.

### Example 4: Testing an In-Vitro Model for Detecting CD44 Elevated Cells Using Fluorescent Probes on Membranes

As described above, in certain embodiments of the present invention, the anti-CD44 fluorescent microsphere conjugates recognize the standard form of CD44 coated on membranes. In this example, it was determined that this imaging agent recognizes CD44 on the surface of cells. In the example, well-characterized cell lines that have been engineered to overexpress or underexpress CD44 were cultured. The cells were plated at low concentration so as to form distinct colonies of various sizes. Then the microsphere conjugates were applied to the colonies, washed, and visualized with UV light. The results were photodocumented and performance of candidate microsphere conjugates were compared. The results were also characterized with confocal microscopy.

### 4.1 Cell Lines

HNSCC cell lines were chosen based on their level of CD44 expression: CAL27 (high), SCC and SCC25 (low). These cell lines are available from ATCC and grow using standard conditions. In prior work [48], CD44 was stably transfected into SCC25 resulting in two clones 2C2-CD44 STD and 3A2-CD44 STD that overexpressed CD44 compared to empty-vector clones 1B1MycHisA and 1B3MycHis. A panel of stable CD44 siRNA clones 3C3- and 4B2-siRNA clones were developed, which showed a reduction in CD44 expression of 90% compared to scramble sequence transfectants. Two overexpressing clones and two under-expressing clones were used in the following experiments.

### 4.2 Culture and Unaided Visual Analysis of Imaging Agents

Cell lines were grown under standard conditions as previously described [48]. The following steps were performed to develop a system to compare candidate anti-CD44 imaging probes.

1) Two CD44 overexpressing clones and two CD44 low clones were grow in culture. Candidate imaging agents (Dragon Green versus Glacial Blue microspheres conjugated with anti-CD44 antibody) were applied in various dilutions using various buffers. Then the cells with applied imaging agents were washed, and visualized with UV light to determine optimal imaging agent concentration, diluent and wash conditions.

2) Once optimal conditions were established, plate each of the four cell line clones in multi-well plates to determine optimal conditions for obtaining single discrete colonies. Visualization were performed with standard microscopy.

3) Using multi-well plates, each of the four clones was plated in triplicate at three different concentrations as determined in step 2 to obtain discrete colonies of various size. Then the initial two imaging agents were used for labeling, and the clones were placed under UV light and visualized with unaided eye. LOD of the agents were determined as measured by lowest number of cells detected. The results were photodocumented.

4) Confocal microscopy was used to compare results with microsphere/antiCD44 conjugates to standard anti-CD44 primary antibody and secondary fluorescent-labeled antibody.

### 4.3 Confocal Analysis of Imaging Agents

After rinsing, cells were grown on chamber slides and blocked for 60 minutes at room temperature, and then incubated as previously described with various anti-CD44 conjugated microspheres or standard primary anti-CD44 antibodies as previously described [48]. Primary antibodies (CD44) are diluted in 1% bovine serum albumin/0.3% Triton X-100. Then, the sections were rinsed and incubated with the secondary antibody conjugated to the fluorescent dye (Alexa Fluor® 488 or Alexa Fluor® 555 from Cell Signaling). The sections were rinsed and incubated with DRAQ5® Dye (Cell Signaling) and then mounted in Slow Fade™ (Invitrogen). Laser confocal microscopy was performed and analyzed using the confocal microscope systems available through the Analytic Imaging Core. Each confocal section was obtained as the average of four frames.

### 4.4 Statistics

The number of cells detected in each triplicate were reported for the 12 experimental conditions (combination of 2 candidate imaging agents, 4 clones, and 3 colony sizes). The estimated LOD for a particular condition is the minimum number of cells detected above 0. The level of fluorescence were scored as 1+, 2+, 3+, etc., in the triplicate under the 12 experimental conditions. T-test or/and the Mann-Whitney U test were used to compare the two imaging agents in the CD44+ cells and in the CD44 low expressors, the agent showing the greatest difference in fluorescence were determined, and these difference were statistically significant.

In certain embodiments, standard culture may not adequately represent the oral cavity and oropharynx. To better represent the target cavity, in one embodiment, the plates were coated with matrigel which simulates the basement membrane. In certain embodiments, a more sensitive agent is provided, where a version of non-toxic Quantum dots is used. As shown in FIG. 4E, the quantum dots have the potential to provide higher signal. In certain embodiments, since it is more challenging to label quantum dots and the quantum dots may have a less robust clinical track record, polystyrene microspheres were chosen. In certain embodiments, antibodies used in the ELISA and lateral flow tests were used in the imaging agent, to ensure recognizing of the same epitope that leads to the positive oral rinse testing. In other embodiments, another anti-CD44 antibody from ATCC, which has been used in clinical trials, were conjugated to fluorescent microspheres or quantum dots to improve the imaging signal. In further embodiments, aptamer specifically binds the target area may be used as the targeting molecule and be included in the imaging agent.

In summary, according to certain embodiments described above, an imaging reagent that produces a signal that is visible to the unaided eye in tissue culture was determined.

### Example 5. Test the System in Tumor Explants from Humans

In Example 4, the anti-CD44 fluorescent microsphere conjugates was determined to recognize native CD44 in a HNSCC cell line. In this example 5, the conjugates were used to identify carcinogenesis in humans using oral cancer specimens and benign tissue from chronic tonsillitis patients ex vivo, following surgical excision of the tissue.

### 5.1 Cases

Patients going to the operating room for surgical removal of an oral cancer, moderate to severe dysplasia or invasive cancer, were consented. Clinical diagnoses of cancer and dysplasia were determined by a trained pathologist. In certain embodiments, subjects with moderate to severe dysplasia and early stage disease (AJCC stage I-II) were enrolled, and no more than 5 late stage tumors (AJCC stage III-IV) were enrolled. Oral rinses were tested for CD44 and protein levels as previously described [12-14] to determine whether CD44 and protein levels were abnormal, indicating a CD44 overexpressing tumor. If elevated, the patient were invited to participate in the next phase of the study.

### 5.2 Controls

Patients going to the OR for removal of chronically inflamed tonsils were consented. Inflammation was reported using standard histopathological criteria. Oral rinses were tested and regardless of level, the excised region using the same system as the cases were examined.

Following surgical excision of the tumor per standard of care, the head and neck surgeon applied the anti-CD44 fluorescent microsphere conjugate to the surface of the tumor and illuminate with the UV light, and then photographed results. In certain embodiments, at least 10 cancer patients and 10 controls were enrolled.

### 5.3 Ex Vivo Imaging

Excised specimens were incubated with the best performing anti-CD44 microsphere conjugates from Example 4 for 10-20 minutes washed in buffer, e.g., phosphate-buffered saline (PBS). Then a UV light were applied and photographs were taken. A grid was applied to the fluorescent image and to the image of the opened specimen so that the coordinates of any areas with altered fluorescence can be recorded and co-localized with the histology. The fresh specimen was then fixed and processed as per normal clinical protocols. Transverse blocks were taken for histological examination throughout the dysplastic or invasive carcinoma segment mapping using the same reference grid as the one applied to the fluorescent images.

### 5.4 Confocal Analysis Ex Vivo

Confocal analysis was performed as described in Example 4. This permits an even more quantitative determination of signal for analysis of differences between normal, benign, disease, dysplasia and invasive carcinoma.

### 5.5 Statistics

For ex vivo experiments, fluorescence levels were measured in 10 cases (in normal, dysplasia, and invasive carcinoma tissues) and in 10 controls (in normal and benign disease tissues). Jittered boxplot were used to visualize the distribution of fluorescence levels in cases (normal vs. dysplasia vs invasive carcinoma tissues) and in controls (normal vs. benign disease tissues). Either the two-tailed t test, if data distribution is approximated normal, and/or the Mann-Whitney U test were applied for comparison of fluorescence values between two independent groups (e.g.: comparison of case/normal tissue vs control/normal tissue, case/invasive carcinoma vs control/benign disease). The Bonferroni method was applied for multiple comparison adjustment, since there are 6 pairwise comparisons of interest. As an illustration of power, for a paired comparison, n=10 provides 80% power to detect an effect size (absolute difference) of 1.37 standard deviations, based on a paired t-test at a two-sided 0.0083 (=0.05/6) significance level. The paired t-test or repeated measures ANOVA or their nonparametric alternatives, the Wilcoxon signed-rank test or Friedman test, were used for comparing fluorescence levels between matched groups. From example, comparison of normal tissue vs. benign disease in controls, or normal tissue vs. dysplasia vs. invasive carcinoma in cases. In addition, the strength and direction of the correlation between fluorescence values in different tissue types were estimated by the Spearman rank-order correlation coefficient in cases and controls. Estimated coefficients and corresponding 95% confidence intervals derived using Fisher's z transformation were reported along with the p value for independence (null correlation). To visualize the relationship between the two potential correlated outcomes, corresponding data were displayed in scatterplots. As an illustration of power, for a two independent group comparison, n=10 per group provides 80% power to detect an effect size (absolute difference) of 1.32 standard deviations, based on a unpaired t-test at a two-sided 5% significance level and assuming equal variance.

In certain embodiments, when certain tissues were not stained well with the microsphere conjugates, different antibodies or QDs may be used.

In summary, among other things, at least one anti-CD44 fluorescent microsphere is provided that distinguishes very well benign and dysplastic or invasive disease with the unaided eye.

### Example 6. Combination of CD44+Total Protein and the Imaging System

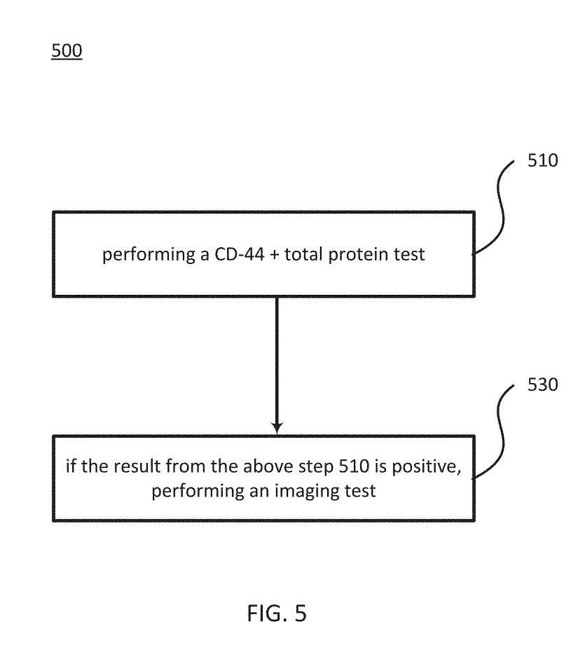

In Example 6, a method is provided to screen and diagnose cancers that is accessible via cavity, such as oral, lung, esophagus, cervical, anal, and colorectal cancers. As shown in FIG. 5, the method includes the steps of 510 and 530.

In step 510, a CD44+total protein test as described in Examples 1 and 2 is performed, so as to detect if a patient has the cancer that is accessible via cavity or has a high risk of that cancer. In the step 510, the concentration of CD44 in a sample from the patient is measured, and the concentration of the total protein in the sample is also determined. In certain embodiments, the result is positive when the concentration of CD44 in the sample is greater than a first pre-determined threshold and the concentration of the total protein in the sample is greater than a second pre-determined threshold.

Then, if the result of the CD44+total protein test 510 is positive, the imaging test 530 is performed to locate the area of the patients having cancer cells.

In certain embodiments, the step of performing an imaging test 530 includes:

providing an imaging agent, the imaging agent comprising:
a fluorescent microsphere; and
a targeting member attached to the fluorescent microsphere;

applying the imaging agent to a testing location of the patient; and

imaging, by an imaging device, the imaging agent in the testing location, so as to detect an amount and a position of the imaging agent in the testing location.

In certain embodiment, the step of applying the imaging agent to the test location of the patient may be performed by rinsing and removing/spitting. For example the patient may perform an oral rinse using the imaging agent, and then spit the imaging agent. The imaging agent left in the mouth of the patient may be specifically bind to locations having cancer cells. The cancer or cancer risk is then determined using the imaging device, where the intensity and location of the imaging agent in the mouth is determined. In one embodiment, the applying the imaging agent step may be performed by spraying the imaging agent to the test location of the patient, and then imaging the intensity and location of the imaging agent.

In certain embodiments, the targeting member is an antibody or an aptamer. In one embodiment, the antibody specifically binds CD44. In certain embodiments, the imaging device is configured to detect light in a range of about 400-700 nm corresponding to the property of the imaging agent. In certain embodiments, the fluorescent microsphere includes a green dye having an excitation maxima at 480 nm and an emission maxima at 520 nm or a blue dye having an excitation maxima at 360 nm and an emission maxima at 450 nm, and a particle size of the fluorescent microsphere is in a range of about 0.2 μm-0.22 μm. In certain embodiments, the fluorescent microsphere has carboxyl groups and the antibody is covalently linked to the fluorescent microsphere through the carboxyl groups.

In this embodiments, whether the imaging test 530 is performed or not is dependent from the result of the CD44+total protein test 510. That is, the CD44+total protein test 510 and the imaging test 530 are performed sequentially when the CD44+total protein test result is positive. In other embodiments, the CD44+total protein test 510 and the imaging test 530 may be independent from each other. For example, a patient may be screen and diagnosed by the imaging test 530 without being tested by the CD44+total protein test 510.

In certain embodiments, the imaging test 530 may use the imaging agent marker. In other embodiment, the imaging agent marker may not be necessary in the imaging test 530, and the imaging test may be performed without applying the imaging agent marker.

In certain embodiments, the imaging test 530 includes fluorescent imaging. In other embodiments, other imaging test 530 may be used together with the CD44+total protein test 510. Such imaging test 530 may include computer tomography (CT) scan, magnetic resonance imaging (MRI) scan, breast MRI, x-rays and other radiographic test, mammography, nuclear medicine scans, ultrasound, etc.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the disclosure pertains without departing from its spirit and scope. Accordingly, the scope of the disclosure is defined by the appended claims as well as the disclosure including drawings.

### REFERENCES

1. Neville B W and Day T A. Oral cancer and precancerous lesions. CA Cancer J Clin. 2002; 52:195-215.
2. SEER Stat Fact Sheets: Oral Cavity and Pharynx; National Cancer Institute. Available at: http://seer.cancer.gov/statfacts/html/oralcay.html
3. Gao J, Panizza B, Johnson N W, Coman S, Clough A R. Basic consideration of research strategies for head and neck cancer. Front Med. 2012; 6: 339-53.
4. Sankaranarayanan R, Mathew B, Jacob B, Thomas G, Somanathan T, Pisani P, Pandey M, Ramadas K, Najeeb K and Abraham E. Early findings from a community-based cluster-randomized, controlled oral cancer screening trial in Kerala, India. Cancer. 2000; 88: 664-73.
5. Sankaranarayanan R, Ramadas K, Thomas G, Muwonge R, Thara S, Mathew B, Rajan B. Trivandrum Oral Cancer Screening Study Group. Effect of screening on oral cancer mortality in Kerala, India: a cluster-randomised controlled trial. Lancet. 2005; 365: 1927-33.
6. Mariotto A B, Yabroff K R, Shao Y, Feuer E J, Brown M L. Projections of the cost of cancer care in the United States: 2010-2020. J Natl Cancer Inst. 2011; 103(2): 117-128. doi: 10.1093/jnci/djq495
7. Jacobson J J, et al. The cost burden of oral, oral pharyngeal, and salivary gland cancers in three groups: commercial insurance, medicare, and Medicaid. Head Neck Oncol. 2012 Apr. 26; 4(1): 15. Available at: http://www.ncbi.nlm.nih.gov/pubmed/22537712
8. Lawless G D: The working patient with cancer: implications for payers and employers. American Health and Drug Benefits. 2009, 2(4): 168-173.
9. Short P, Moran J, Rajeshwari P. Medical expenditures of adult cancer survivors aged <65 years in the United States. Cancer. 2011, 117(12): 2791-2800.
10. Ellison M D, Campbell B H. Screening for cancers of the head and neck: addressing the problem. Surg. Oncol. Clin. N. Am. 1999; 8: 725-34.
11. Franzmann E J, Reategui E P, Pedroso F, Pernas F G, Karakullukcu B M, Carraway K L, Hamilton K, Singal R, Goodwin W J. Soluble CD44 is a potential marker for the early detection of head and neck cancer. Cancer Epidemiol. Biomarkers Prev. 2007; 16: 1348-1355.
12. Pereira L M, Adebisi I N, Perez A, Wiebel M, Reis I, Duncan R, Goodwin W J, Hu J J, Lokeshwar V B, Franzmann E J. Salivary markers and risk factor data: A multivariate modeling approach for head and neck squamous cell carcinoma detection. Cancer Biomarkers. 2012; 10: 241-49.
13. Franzmann E J, Reategui E P, Carraway K L, Hamilton K L, Weed D T, Goodwin W J. Salivary soluble CD44: a potential molecular marker for head and neck cancer. Cancer Epidemiol. Biomarkers Prev. 2005; 14: 735-739.
14. Franzmann E J, Reategui E, Mateus Pereira L H, Pedroso F, Joseph D, Allen G O, Hamilton K, Reis I, Duncan R, Goodwin W J, Hu J J, Lokeshwar V B. Salivary protein and solCD44 levels as a potential screening tool for early detection of head and neck squamous cell carcinoma. Head Neck. 2012 May; 34(5): 687-95.
15. Day G L and Blot W. Second primary tumors in patients with oral cancer. Cancer. 1992; 70:14-19.
16. Ferlay J, Shin H R, Bray F, Forman D, Mathers C, Parkin D M. Estimates of worldwide burden of cancer in 2008: GLOBOCAN 2008. Int J Cancer. 2010; 127: 2893-917.
17. American Cancer Society. Cancer Facts and Figures 2014. Atlanta: American Cancer Society 2014.
18. Winn D M, Blot W J, Shy C M, Pickle L W, Toledo A and Fraumeni J F. Snuff dipping and oral cancer among women in the Southern United States. N. Engl. J. Med. 1981; 304: 745-9.
19. D'Souza G, Kreimer A R, Viscidi R, Pawlita M, Fakhry C, Koch W M, Westra W H, Gillison M L. Case-control study of human papillomavirus and oropharyngeal cancer. N. Engl. J. Med. 356: 1944-56, 2007.
20. Muscat J E and Wynder E L. Tobacco, alcohol, asbestos, and occupational risk factors for laryngeal cancer. Cancer. 1992; 69: 2244-51.
21. Blot W J, McLaughlin J K, Winn D M, Austin D F, Greenberg R S, Preston-Martin S, Bernstein L, Schoenberg J B, Stemhagen A and Fraumeni J F. Smoking and drinking in relation to oral and pharyngeal cancer. Cancer Res. 1988; 48: 3282-7.
22. Burch J D, Howe G R, Miller A B and Semenciw R. Tobacco, alcohol, asbestos, and nickel in the etiology of cancer of the larynx: a case-control study. J. Natl. Cancer Inst. 1981; 67: 1219-24.
23. Johnson N. Tobacco use and oral cancer: a global perspective. J. Dental Edu. 2001; 65: 328-339.
24. Balaram P, Sridhar H, Rajkumar T, Vaccarella S, Herrero R, Nandakumar A, Ravichandran K, Ramdas K, Sankaranarayanan R, Gajalakshmi V, Munoz N and Franceschi S. Oral cancer in southern India: the influence of smoking, drinking, paan-chewing and oral hygiene. Int. J. Cancer. 2002; 98: 440-45.
25. Lewin F, Norrell S E, Johansson H, Gustaysson P, Wennerberg J, Biorklund A and Rutqvist L E. Smoking tobacco, oral snuff and alcohol in the etiology of squamous cell carcinoma of the head and neck. Cancer. 1998; 82: 1367-75.
26. Mashberg A, Boffetta P, Winkelman R and Garfinkel L. Tobacco smoking, alcohol drinking, and cancer of the oral cavity and oropharynx among U.S. veterans. Cancer. 1993; 72: 1369-75.
27. Talamini R, La Vecchia C, Levi F, Conti E, Favero A and Franceschi S. Cancer of the oral cavity and pharynx in nonsmokers who drink alcohol and in nondrinkers who smoke tobacco. J. Natl. Cancer Inst. 1998; 90: 1901-3.
28. Oral Cancer Facts; Oral Cancer Foundation; Available at: http://oralcancerfoundation.org/facts/index.htm
29. The global economic cost of cancer. American Cancer Society, Inc. 2010.
30. Mehrotra R, Singh M, Thomas S, Nair P Pandya S, Nigam N S, Shukla P. A cross-sectional study evaluating chemiluminescence and autofluorescence in the detection of clinically innocuous precancerous and cancerous oral lesions. JADA. 2010; 141(2): 151-156.
31. Su W W, Yen A M, Chiu S Y and Chen T H. A Community-based RCT for Oral Cancer Screening with Toluidine Blue. J Dent Res. 2010; 89: 933-937.
32. Shankar A A, Routray S. Trends in salivary diagnostics—a 5-year review of oral oncology (2007-2011). Oral Oncol. 2012; 48: e22-3.
33. Li Y, St. John M A, Zhou X, Kim Y, Sinha U, Jordan R C, et al. Salivary transcriptome diagnostics for oral cancer detection. Clin. Cancer Res. 2004; 10: 8442-8450.
34. Park N J, Zhou H, Elashoff D, Henson B S, Kastratovic D A, Abemayor E, et al. Salivary microRNA: discovery, characterization, and clinical utility for oral cancer detection. Clin. Cancer Res. 2009; 15: 5473-5477.
35. Hu S, Arellano M, Boontheung P, Wang J, Zhou H, Jiang J, et al. Salivary proteomics for oral cancer biomarker discovery. Clin. Cancer Res. 2008; 14: 6246-6252.
36. Carvalho A L, Jeronimo C, Kim M M, Henrique R, Zhang Z, Hoque M O, Chang S, Brait M, Nayak C S, Jiang W W, Claybourne Q, Tokumaru Y, Lee J, Goldenberg D, Garrett-Mayer E, Goodman S, Moon C S, Koch W, Westra W H, Sidransky D, Califano J A. Evaluation of promoter hypermethylation detection in body fluids as a screening/diagnosis tool for head and neck squamous cell carcinoma. Clin Cancer Res. 2008; 14: 97-107.
37. Elashoff D, Zhou H, Reiss J, Wang J, Xiao H, Henson B, Hu S, Arellano M, Sinha U, Le A, Messadi D, Wang M, Nabili V, Lingen M, Morris D, Randolph T, Feng Z, Akin D, Kastratovic D A, Chia D, Abemayor E, Wong D T. Prevalidation of salivary biomarkers for oral cancer detection. Cancer Epidemiol Biomarkers Prev. 2012; 21: 664-72.
38. Pepe M S, Etzioni R, Feng Z, Potter J D, Thompson M L, Thornquist M, Winget M, Yasui Y. Phases of biomarker development for early detection of cancer. Journal of the National Cancer Institute. 2001; 93(14): 1054-61.
39. Lingen M W, Kalmar J R, Karrison T, Speight P M. Critical evaluation of diagnostic aids for the detection of oral cancer. Oral Oncol. 2008; 44:10-22.
40. Balevi B. Assessing the usefulness of three adjunctive diagnostic devices for oral cancer screening: a probabilistic approach. Community Dent Oral Epidemiol. 2011; 39:171-6.
41. http://www.newswise.com/articles/double-vision-new-imaging-agent-improves-surgeon-s-ability-to-find-remove-cancer
42. Bao, Liwei, et al. Preclinical development of a bifunctional cancer cell homing, PKCε inhibitory peptide for the treatment of head and neck cancer. Cancer Research. 2009; 69: 5829-5834.
43. Baeten, John, et al. Molecular Imaging of Oral Premalignant and Malignant Lesions Using Fluorescently Labeled Lectins. Translational Oncology. 2014; 7: 213-220.
44. Bird-Lieberman, Elizabeth L., et al. Molecular imaging using fluorescent lectins permits rapid endoscopic identification of dysplasia in Barrett's esophagus. Nature Medicine. 2012; 18(2): 315-321.
45. Schmidt, Carsten, et al. Nano- and microscaled particles for drug targeting to inflamed intestinal mucosa-A first in vivo study in human patients. Journal of Controlled Release. 2013; 165(2): 139-145.
46. Lauren, Mark, and Frederick McIntyre. 4D Clinical Imaging for Dynamic CAD. International Journal of Dentistry. 2013.
47. Charmot, Dominique. Non-systemic drugs: a critical review. Current Pharmaceutical Design. 2012; 18(10): 1434.
48. Perez A, Neskey D, Wen J, Pereira L, Reategui E P, Goodwin J W, Carraway K L, and Franzmann E J. CD44 interacts with EGFR and promotes Head and Neck Squamous Cell Carcinoma (HNSCC) initiation and progression. Oral Oncol. 2013; 49: 306-13

What is claimed is:

1. A system for testing a biological subject, comprising:
an imaging device; and
an imaging agent detectable by the imaging device, comprising:
a fluorescent microsphere; and
a targeting member attached to the fluorescent microsphere.

2. The system of claim 1, wherein the targeting member is an antibody or an aptamer.

3. The system of claim 2, wherein the antibody specifically binds CD44.

4. The system of claim 1, wherein the imaging device is configured to detect light in a range of about 400-700 nm corresponding to a property of the imaging agent.

5. The system of claim 4, wherein the fluorescent microsphere comprises a green dye having an excitation maxima at 480 nm and an emission maxima at 520 nm or a blue dye having an excitation maxima at 360 nm and an emission maxima at 450 nm.

6. The system of claim 1, wherein a particle size of the fluorescent microsphere is in a range of 0.2 μm-15 μm.

7. The system of claim 6, wherein the particle size of the fluorescent microsphere is in a range of 0.2 μm-0.22 μm.

8. The system of claim 1, wherein the fluorescent microsphere has carboxyl groups and the targeting member is covalently linked to the fluorescent microsphere through the carboxyl groups.

9. The system of claim 1, wherein the fluorescent microsphere is made of latex and the targeting member is adsorbed to a surface of the fluorescent microsphere through hydrophobic attractions.

10. The system of claim 1, wherein during testing of the biological subject, the imaging agent is used to rinse the mouth of a patient and is then spit by the patient, and the imaging agent bound to a target location in the mouth is detected by the imaging device.

11. The system of claim 10, wherein during testing of the biological subject, an ultraviolet light or another light that is capable of exciting the fluorescent microsphere to emit in the visible range is applied to the mouth of the patient.

12. The system of claim 1, wherein during testing of the biological subject, the imaging agent is sprayed to a test location of a patient, and then imaged using the imaging device.

13. A method of testing a biological subject, comprising:
providing an imaging agent, the imaging agent comprising:
a fluorescent microsphere; and
a targeting member attached to the fluorescent microsphere;
applying the imaging agent to a testing location of a patient; and
imaging, by an imaging device, the imaging agent in the testing location, so as to detect an amount and a position of the imaging agent in the testing location.

14. The method of claim 13, wherein the targeting member is an antibody or an aptamer.

15. The method of claim 14, wherein the antibody specifically binds CD44.

16. The method of claim 13, wherein the imaging device is configured to detect light in a range of about 400-700 nm corresponding to a property of the imaging agent.

17. The method of claim 16, wherein the fluorescent microsphere comprises a green dye having an excitation maxima at 480 nm and an emission maxima at 520 nm or a blue dye having an excitation maxima at 360 nm and an emission maxima at 450 nm.

18. The method of claim 13, wherein a particle size of the fluorescent microsphere is in a range of 0.2 μm-15 μm.

19. The method of claim 13, wherein the particle size of the fluorescent microsphere is in a range of 0.2 μm-0.22 μm.

20. The method of claim 13, wherein the fluorescent microsphere has carboxyl groups and the targeting member is covalently linked to the fluorescent microsphere through the carboxyl groups.

21. The method of claim 13, wherein the fluorescent microsphere is made of latex and the targeting member is adsorbed to a surface of the fluorescent microsphere through hydrophobic attractions.

22. The method of claim 13, wherein the step of applying the imaging agent to the test location of the patient comprises rinsing the test location with the imaging agent or spray the imaging agent on the test location.

23. A method of screening and diagnose a cancer that is accessible via cavity of a patient, comprising the steps of:
performing a test measuring a biomarker selected from a group consisting of at least CD44, total protein, or both CD44 and total protein; and
if a result from the performing the test is positive, performing an imaging test.

24. The method of claim 23, where the step of performing the test comprises:
measuring a concentration of CD44 in a sample from the patient; and
measuring a concentration of total protein in the sample.

25. The method of claim 24, where the result of the step of performing the test is positive when the concentration of CD44 in the sample is greater than a first pre-determined threshold and the concentration of the total protein in the sample is greater than a second pre-determined threshold.

26. The method of claim 23, where the step of performing an imaging test comprises:
providing an imaging agent, the imaging agent comprising:
a fluorescent microsphere; and
a targeting member attached to the fluorescent microsphere;
applying the imaging agent to a test location of the patient; and
imaging, by an imaging device, the imaging agent in the testing location, so as to detect an amount and a position of the imaging agent in the testing location.

27. The method of claim 26, wherein the step of applying the imaging agent to the test location of the patient comprises rinsing the test location with the imaging agent or spray the imaging agent on the test location.

28. The method of claim 26, wherein the targeting member is an antibody or an aptamer.

29. The method of claim 28, wherein the imaging device is configured to detect light in a range of about 400-700 nm corresponding to a property of the imaging agent.

30. The method of claim 29, wherein
the antibody specifically binds CD44;
the fluorescent microsphere comprises a green dye having an excitation maxima at 480 nm and an emission maxima at 520 nm or a blue dye having an excitation maxima at 360 nm and an emission maxima at 450 nm; and
a particle size of the fluorescent microsphere is in a range of about 0.2 μm-0.22 μm.

31. The method of claim 26, wherein the fluorescent microsphere has carboxyl groups and the antibody is covalently linked to the fluorescent microsphere through the carboxyl groups.
